(19) United States
(12) Patent Application Publication
(10) Pub. No.: US 2017/0252140 A1
(43) Pub. Date: Sep. 7, 2017

(54) DENTAL APPLIANCE HAVING SENSING CAPABILITIES

(71) Applicant: TECHDERM, LLC, Ossining, NY (US)

(72) Inventors: Shaun P. MURPHY, Orlando, FL (US); Charles E. MURPHY, Sarasota, FL (US); Bryce A. WAY, San Jose, CA (US); Richard JOHNSON, Briarcliff Manor, NY (US); Amin H. MIRZAAGHAEIAN, Fremont, CA (US)

(21) Appl. No.: 15/517,385

(22) PCT Filed: Oct. 14, 2015
(86) PCT No.: PCT/US2015/055606

§ 371 Date: Apr. 6, 2017

Related U.S. Application Data
(60) Provisional application No. 62/063,843, filed on Oct. 14, 2014.

(51) Int. Cl. A61C 19/04; A61B 5/00; A61C 7/08

(52) U.S. Cl. CPC A61C 19/04; A61C 7/08; A61B 5/4833; A61C 2204/005

(57) ABSTRACT

A dental usage monitoring system having a dental appliance, a sensor unit having at least one sensor attached to the dental appliance, a power supply attached to the dental appliance, and an analyzer. The at least one sensor attached to the dental appliance is configured to collect data related to usage of the dental appliance. An analyzer in communication with the sensor and is configured to determine usage of the dental appliance based upon the collected data. The dental usage monitoring system can include a base module configured to receive collected data, transmit data to the analyzer, and recharge the sensor unit. The base module can be further configured to calibrate the collected data. The sensor unit can be embedded in the dental appliance or coupled to the dental appliance. The sensor unit may be encased in a protective coating.

### CROSS REFERENCE TO RELATED APPLICATIONS

This application claims priority to U.S. Provisional Application No. 62/063,843 filed on Oct. 14, 2014, entitled “SENSOR AND METHODS OF TRANSFORMING IMPRECISE DATA INTO DISCRETE DECISIONS,” the entirety of which is incorporated by reference.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present disclosure is directed to novel dental appliance and accessories that are able to detect and transmit either a physiological parameter or a physical parameter when worn.

### BACKGROUND

Orthodontic treatments are the leading way to straighten or re-position teeth to not only improve the appearance of teeth, but also how they function. The typical treatment includes use of bonded structures (such as braces) or removable structures (such as Invisalign®). Typical course of treatment for both the bonded or removable type of braces can take up to a couple of years. In most cases, when orthodontic treatments are completed, a patient is still required to wear retainers for an additional length of time.

Retainers help to maintain the newly re-positioned teeth in their current positions, allowing the teeth to become accustomed to their new positions. Retainers also prevent the newly re-positioned teeth from moving back to their pre-adjustment positions. Initially, patients may be required to wear retainers for the entire day except when eating. The frequency with which a patient is required to wear retainers generally decreases over time to several hours a day and at night or even only at night.

When patients fail to wear their retainers for the prescribed amount of time, the effect of the orthodontic treatment on teeth tends to undo itself, and the teeth may stray back to their original positions. The “undoing” effect of not properly using retainers or not using retainers for the requisite amount of time means money wasted. The present cost of orthodontic treatment ranges $4,000 to $10,000. While most adults tend to be more cognizant of the cost of the orthodontic treatment and follow the recommended wear time for their retainers, children and adolescents often forgot or chose not to wear their retainer for the prescribed amount of time.

As mentioned above, failing to wear retainers for the prescribed amount of time correlates to a lengthier treatment period and, often times, more costly treatment. In the past, when patients, especially children and adolescents, fail to wear their retainer, there was no way for either their parents or orthodontics to know how long the children/adolescents actually wore their retainer. In some cases, parents blame orthodontists for the failure of the orthodontic treatment because they are unaware that their children have not properly wearing their retainers. There is currently no product on the market that is able to monitor a patient's retainer wear time.

Thus, there exists a need in the orthodontic field for a retainer device that is able to sense, monitor, and report back on a wearer's usage.

### SUMMARY OF THE DISCLOSURE

The present invention relates to devices, methods and systems for monitoring use of a dental appliance. More particularly, the present invention is directed towards monitoring usage of orthodontic maintenance appliances.

Orthodontic maintenance appliances, such as retainers, are worn by most patients once the active portion of the orthodontic treatment has been completed and the maintenance portion commences. The lasting effects of the orthodontics treatment is directly proportional to the amount of time that the patients wears their retaining device.

In general, in one embodiment, a dental usage monitoring system includes a dental appliance, at least one sensor attached to the dental appliance, a power supply attached to the dental appliance, and an analyzer. The at least one sensor attached to the dental appliance is configured to collect data related to usage of the dental appliance. A power supply attached to the dental appliance is configured to operate the sensor using a current of less than 1 milliamp. An analyzer is in communication with the sensor and configured to determine usage of the dental appliance based upon the collected data.

This and other embodiments can include one or more of the following features. The data can be voltage data. The dental usage monitoring system can further include a base module configured to receive the collected data, determine temperature readings from the voltage data, and transmit the temperature readings to the analyzer. The base module can be further configured to calibrate the collected data. The base module can be further configured to recharge the power supply on the dental appliance. The power supply can be a supercapacitor. The sensor can be embedded in the dental appliance. The sensor can be attached to the dental appliance in a region that corresponds to an open or empty pocket between a user's teeth and a buccal region of the user's mouth when in use. The sensor can further include a protective coating therearound. The protective coating can include a silicon-based compound, a ceramic-based compound, a plastic material, such as a resin, a resinoid, a polymer, a cellulose derivative, a casein material, and/or a protein, or a composite material. The data can be temperature, motion, position, force, pressure, pH, oxygen concentration, carbon dioxide concentration, bacteria count, heartbeat, or presence of arrhythmias. The dental appliance can be a retainer. The analyzer can be in wireless communication with the sensor, the base module, or both.

In general, in one embodiment, a dental usage monitoring system includes a dental appliance, at least one sensor attached to the dental appliance, a base module, and an analyzer. The at least one sensor attached to the dental appliance is configured to detect data related to use of the dental appliance. A base module is configured to couple with the dental appliance to receive the detected data from the sensor and calibrate the data. An analyzer in communication with the base module is configured to determine usage of the dental appliance based upon the calibrated data.

This and other embodiments can include one or more of the following features. The base module can further include a base sensor. The base module can be configured to calibrate the collected data based upon a comparison with data obtained from the based sensor. The data can be voltage data. The base module can be further configured to determine temperature readings from the voltage data. The sensor can be configured to transmit data to the base module only when the sensor is a set distance from the base module. The base module can further be configured to recharge a power supply for the sensor. The sensor can be embedded in the dental appliance. The sensor can be attached to the dental appliance in a region that corresponds to an open or empty pocket between a user's teeth and a buccal region of the user's mouth when in use. The sensor can further include a protective coating therearound. The protective coating can include a silicon-based compound, a ceramic-based compound, a plastic material, such as a resin, a resinoid, a polymer, a cellulose derivative, a casein material, and/or a protein, or a composite material. The data is temperature, motion, position, force, pressure, pH, oxygen concentration, carbon dioxide concentration, bacteria count, heartbeat, or presence of arrhythmias. The dental appliance can be a retainer. The analyzer can be in wireless communication with the sensor. The sensor can be in wireless communication with the base module.

In general, in one embodiment, a dental usage monitoring system includes a dental appliance, at least one sensor attached to the dental appliance, and an analyzer. The at least one sensor attached to the dental appliance is configured to collect data related to usage of the dental appliance at discrete timepoints. An analyzer in communication with the sensor is configured to determine total usage of the dental appliance based upon the data collected at discrete timepoints.

This and other embodiments can include one or more of the following features. The sensor can be configured to take measurements only when activated. The sensor can be configured to be activated based upon removal from a base module. The timepoints can be at least 5 minutes apart. The analyzer can be configured to use a decision tree classifier to process variability in the data received, and to determine the total usage. The sensor can be configured to transmit data to the base module only when the sensor is a set distance from the base module. The base module can further be configured to recharge a power supply for the sensor. The sensor can be embedded in the dental appliance. The sensor can be attached to the dental appliance in a region that corresponds to an open or empty pocket between a user's teeth and a buccal region of the user's mouth when in use. The sensor can further include a protective coating therearound. The protective coating can include a silicon-based compound, a ceramic-based compound, a plastic material, such as a resin, a resinoid, a polymer, a cellulose derivative, a casein material, and/or a protein, or a composite material. The data can be temperature, motion, position, force, pressure, pH, oxygen concentration, carbon dioxide concentration, bacteria count, heartbeat, or presence of arrhythmias. The dental appliance can be a retainer. The analyzer can be in wireless communication with the sensor. The sensor can be in wireless communication with the base module.

In general, in one embodiment, a dental usage monitoring system includes a dental appliance, at least one sensor attached to the dental appliance, and a global positioning system attached to the dental appliance. The at least one sensor attached to the dental appliance is configured to collect data related to usage of the dental appliance.

This and other embodiments can include one or more of the following features. The dental usage monitoring system can further include a base module that is configured to couple with the dental appliance to receive the collected data or receive information from the global positioning system.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a high level block diagram depicting components of a dental usage monitoring system.

FIG. 2 is a more detailed schematic of the dental usage monitoring system.

FIG. 3 shows a sensor placed next to a United States ten cent coin for size comparison.

FIG. 4A and 4B are schematics of sensor components.

FIG. 5 is a graph showing testing of a temperature sensor.

FIG. 6 shows a first embodiment of the dental usage monitoring system showing the sensor attached to a first example of a dental appliance.

FIG. 7 shows a second embodiment of the dental usage monitoring system showing the sensor attached to a second example of a dental appliance.

FIG. 8 shows an embodiment of a base module.

FIG. 9 is a picture showing the second example of the dental appliance positioned on the base module as it would be when charging or transferring data.

FIG. 10A shows internal electronic components of the base module.

FIG. 10B shows the internal electronics components of the base module with the sensor placed in a region optimal for charging and transferring data.

FIG. 11 is a diagram showing the relation and function of a sensor unit (parameter to be detected), with the base module and the analyzer.

FIG. 12 is a diagram showing the relation between the base module, analyzer, and the user.

### DETAILED DESCRIPTION

Described herein are systems, devices, and related programs for monitoring dental appliance usage. In general, the dental usage monitoring system includes a dental appliance, a sensor coupled to the dental appliance, a data reader and charging base module, and related programs for calibrating and analyzing data recorded. In general, the dental usage monitoring system is used to track usage of the dental appliance, particularly, an orthodontic retainer. The data recorded can be received, analyzed, processed, and displayed dental providers and the wearer and their caregivers can track the dental appliance usage.

The proposed device or other embodiments of such device are able to attach/integrate with any and all dental or orthodontic mouthpieces, sizing mouthpieces, dental sleep appliances, palatal expanders, mouth guards, sport mouth guards, dental casting mouthpiece, dental and orthodontic retainer, or orthodontic and dental aligner.

Advantageously, the sensor unit and methods described herein include the use of very small, inexpensive, and low-power sensors in an application that would normally require a much more robust and larger sensor.

In general, the dental usage monitoring system described herein is able to receive and transmit dental-related information that will ultimately be analyzed. The system includes a dental appliance, a sensor unit for detecting one or more environmental parameter, and an analyzer that is able to transform imprecise, raw data into reliable data that can be correlated with some characteristic of the wearer or related to the wearer's course of dental treatment, such as time that the dental appliance is worn. The sensor unit may be coupled to or embedded in the dental appliance. In the former case, the sensor unit may be coupled to the dental appliance in a region that corresponds to the “dead zone” or and open region, a region between the wearer's molars and his buccal.

The sensor unit can be covered by a protective coating, such as a silicon-based compound, a ceramic-based compound, a plastic material, such as a resin, a resinoid, a polymer, a cellulose derivative, a casein material, and/or a protein, or a composite material.

The sensors described herein can be used to measure an environmental parameter, i.e., a measurable parameter within the wearer's mouth. Parameters include, but are not limited to temperature, motion, force, pressure, pH, oxygen concentration, carbon dioxide concentration, bacteria count, heartbeat, or arrhythmias.

In some embodiments, also included in the dental usage monitoring system is a base module. The base module functions to wirelessly recharge the sensor unit and to retrieve and storage raw data that the sensor unit has detected and stored. The base module can then transmit the raw data to the analyzer. Transmission may be either wirelessly through a local network, or through a cable attached to a USB port on the base module.

The monitoring device described herein is able to collect and transmit data related to use of the dental appliance. The usage monitoring system includes a dental appliance and a sensor. Dental appliance can refer to dental devices that a user may remove periodically for activities such as eating or is on a schedule of wear where the use may only be a few hours during the day or only at night. A dental appliance can also be a semi-permanent dental devices. More specifically, the dental appliance conceived of for use in the usage monitoring system can be an orthodontic retainer.

The sensor is coupled to the dental appliance and configured to measure, retain, and transmit information related to use of the dental appliance. Sensor may refer to an object or device that is able to detect an event or parameter and change in the event or parameter and provide a corresponding output. More specifically, the sensors conceived of for use in the usage monitoring system can be related to being able to detect a parameter that can be correlated with usage of the dental appliance.

In some instances, the sensor is embedded in the dental appliance. In the case of a retainer, the sensor may be embedded in a molded palate in the portion of the retainer corresponding to the palate of the user's mouth. At this location, the added thickness to the retainer from the sensor is minimally intrusive. In other instances the sensor may be coupled to the dental appliance at a location least intrusive to the user, such as between the wearer's teeth and his cheeks.

The sensor may also include a protective coating. The protective coating not only protects the components of the sensor from the moisture and degrading elements within the wearer's mouth in the design where the sensor is coupled to and not embedded in dental appliance. The protective coating also protects the wearer from any harmful effects of the components contained within the sensor. Even in when the sensor is embedded within the dental appliance, the protective coating provides an extra layer of protection during the embedding process.

As previously mentioned, the sensor is able to detect a parameter associated with use of the dental appliance. In some instances, the parameter is a biological parameter associated with the wearer. This may include measuring a body temperature, wearer's heartbeat, pH, or a concentration of a particular molecular species such as oxygen or carbon dioxide. In other instances, the sensor may measure a physical parameter such as motion and pressure/force.

The usage monitoring system also includes a base module for charging the sensor and receiving data from the sensor. Because the sensor is intended to be used inside a mouth, a relatively moist environment, expose electronics is undesirable. In some examples, then sensor may be able to wireless charge when it is placed on the base module. The base module may also be able to download data collected by the sensor when the sensor is in close proximity to the base module. The base module is then able to transmit the collected data to a user interface on a telecommunication device, where the data can be aggregated and viewed. The base module may also include some user interfaces that allow a user to control the base module's function. The base module may also include audio signal to alert the wearer when the sensor has completely charged. Finally, the base module may include a cord and plug for electrically connecting to a wall outlet.

There may be programs and applications associated with the dental usage monitoring system. Programs for a computer or laptop or corresponding application for smart devices are able to receive data downloaded from the sensor. These programs are then configured to process the data into an easily viewable form for the dental provider or other interested party to review. The data may be presented in graphical form, tabular form, and so forth.

More specifically, methods of calibrating and transforming imprecise sensor data into reliable data that can measure changes in phase and frequency to produce high confidence decisions, such as the amount of time that a dental appliance is in a patient's mouth.

Also disclosed herein are methods of using the dental usage monitoring system. The methods include signaling to the sensor to switch between a sleep mode and an awake mode based on a predetermined condition being met. The sensor is then able to switch back to a sleep mode upon a second set of conditions being present.

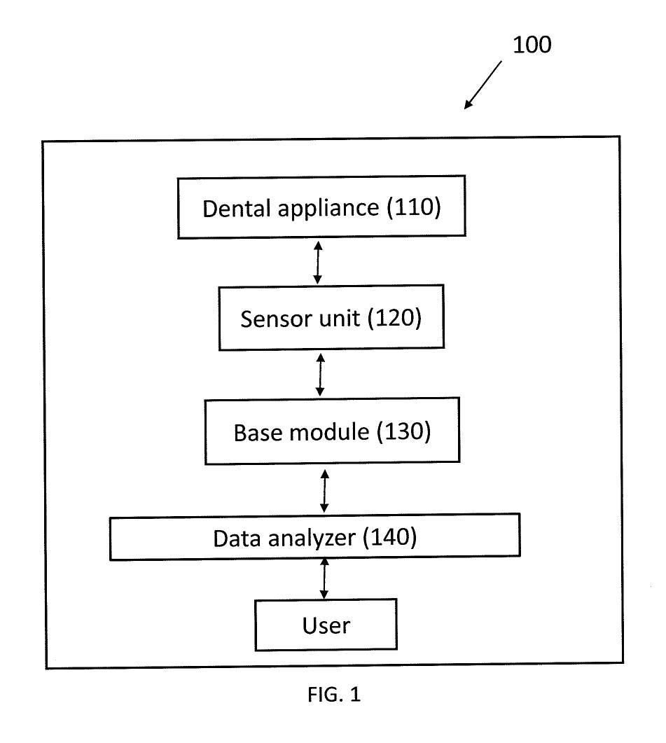

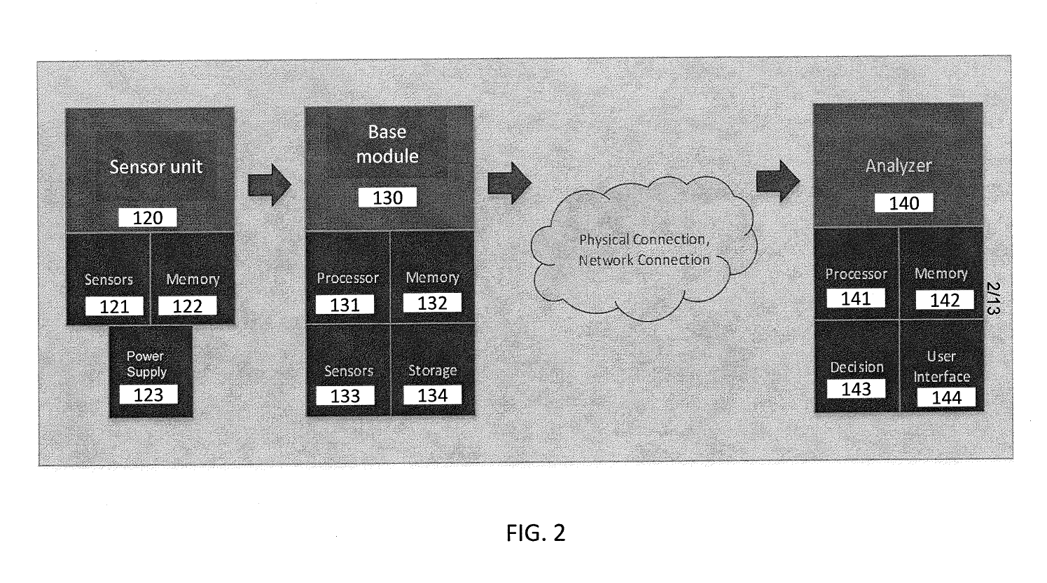

FIGS. 1 and 2 are block diagrams of a dental usage monitoring system 100. As FIG. 1 shows, the dental usage monitoring system 100 includes a dental appliance 110, a sensor system 120, a base module 130, and a data analyzer 140. The sensor unit 120 is physically coupled to the dental appliance 110. The base module 130 is able to wirelessly communication with the sensor unit 120 to retrieve data recorded and is able to send data (either raw or partially processed) to the analyzer 140. The analyzer 140 is then able to process the raw or partially processed data into easy to read data which can be in the form of a graph or table. Also, the sensor unit 120, the base module 130, and the analyzer 140 may be configured to transform imprecise recorded data and convert it to more precise data using discrete decision algorithms. The dental usage monitoring system 100 can be used to measure a parameter within the wearer's mouth. In some examples, the measured parameter may be correlated with the amount of time that the wearer is wearing the dental appliance 110. The measured parameter may be a biological value that is measurable within the wearer's mouth.

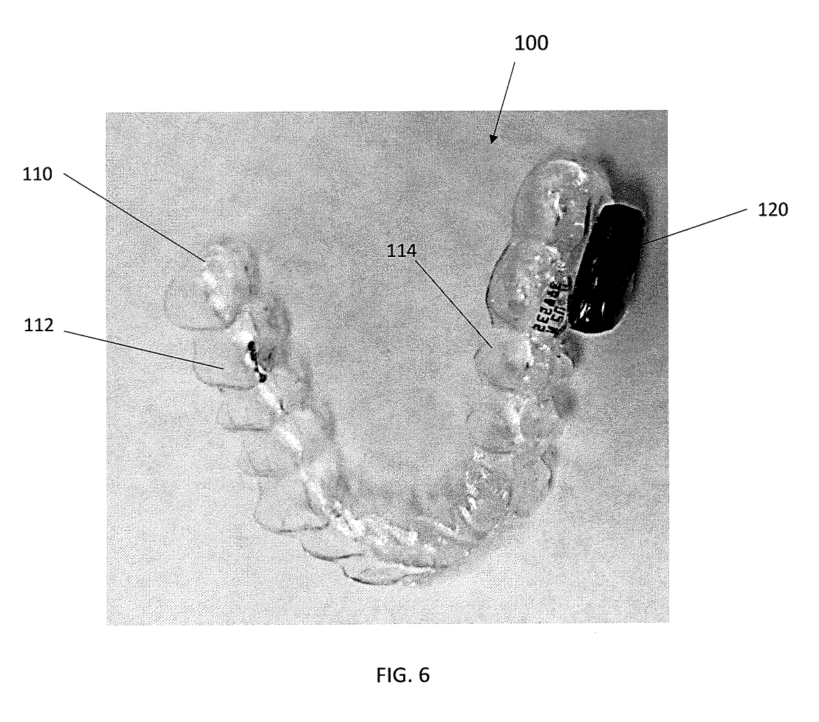

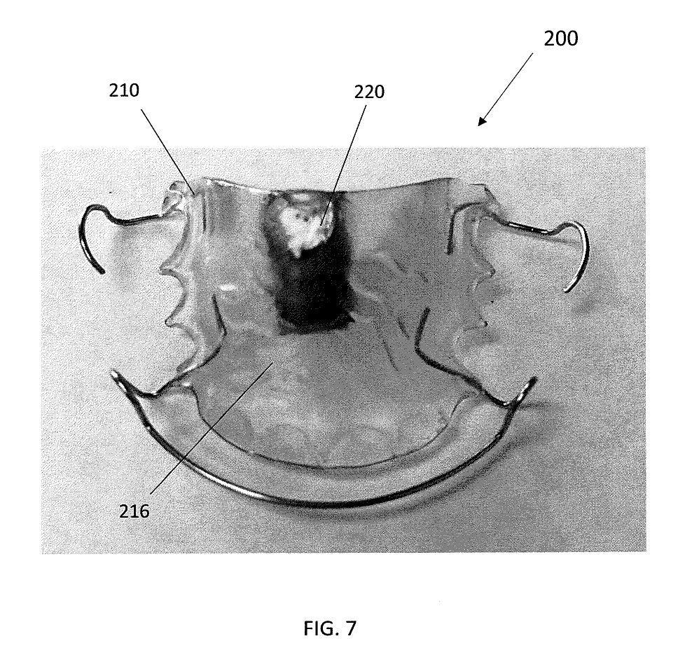

Referring to FIGS. 6 and 7, the dental appliance 110 can be a device that is used or worn within the mouth of the wearer. The dental appliance 110 may include, for example, a device used in preventing trauma to the teeth or other part of the wearer's mouth or in treating symptoms of a dental or an oral condition (such as a mouth guard for protecting teeth during athletics, night guard during sleep, or snoring devices for addressing sleep apnea). The dental appliance 110 can also be a device used for addressing dental loss or disease (such as dental prosthesis, dentures or partial dentures). The dental appliance 110 may also be a device that is used to correct a pre-existing dental condition (orthodontic braces) and/or maintain teeth in correct position post-adjustment (such as orthodontic retainers). The dental appliance 110 is primarily described herein as being directed to maintaining teeth during and after an orthodontic regimen. However, it should be understood that the dental usage monitoring system can include or be used with other dental appliances mentioned above.

In some embodiments, the dental usage monitoring system contains a sensor for measuring one parameter, but in other embodiments, the dental usage monitoring system may contain more than one sensor and their associated components for detecting more than one parameter.

Referring still to FIGS. 6 and 7, the dental appliance 110 can be a device configured to be removed by the wearer. Further, the dental appliance 110 can be one that, if not worn for the prescribed regimen, may result in lengthier period of treatment and even reversal of the desired treatment results over time. Two different examples of a dental appliance 110 are shown in FIGS. 6 and 7. The dental appliance 110 shown in FIG. 6 is a custom aligner synonymous with the Invisalign® regimen of repositioning teeth, as well as the corresponding retainer device. The dental appliance 210 shown in FIG. 7 is an orthodontic retainer (the Hawley retainer) having a metal wire that typically surrounds the six anterior teeth and keeps teeth in place.

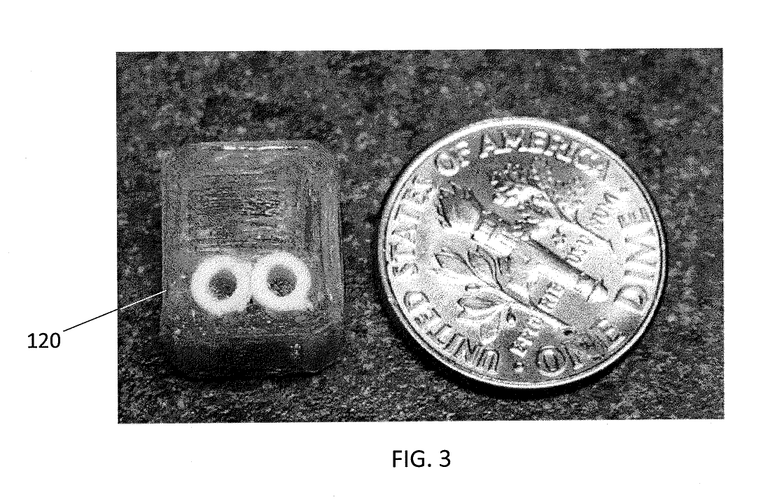

As previously mentioned, one or more sensor units 120 can be coupled to the dental appliance 110. FIG. 3 shows an example of the relative size of sensor unit 120 as compared to a United States ten cent (dime) coin. In some embodiments, the sensor unit 120 may cover an area (length and width) of no larger than 1 cm² or 0.5 cm². Further, in one embodiment, the sensor is less than 4 mm thick, such less than 3 mm thick.

In some embodiments, the sensor unit 120 can be encapsulated, as shown in FIG. 3. For example, to encapsulate the sensor unit 120, it may be dipped in a biologically compatible resin, polymer, nanomaterials, or silicon or carbon-based materials.

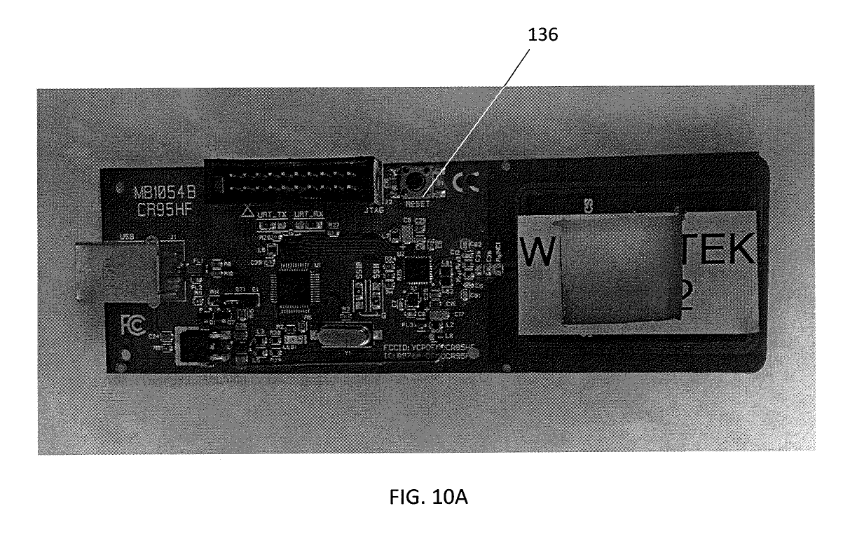

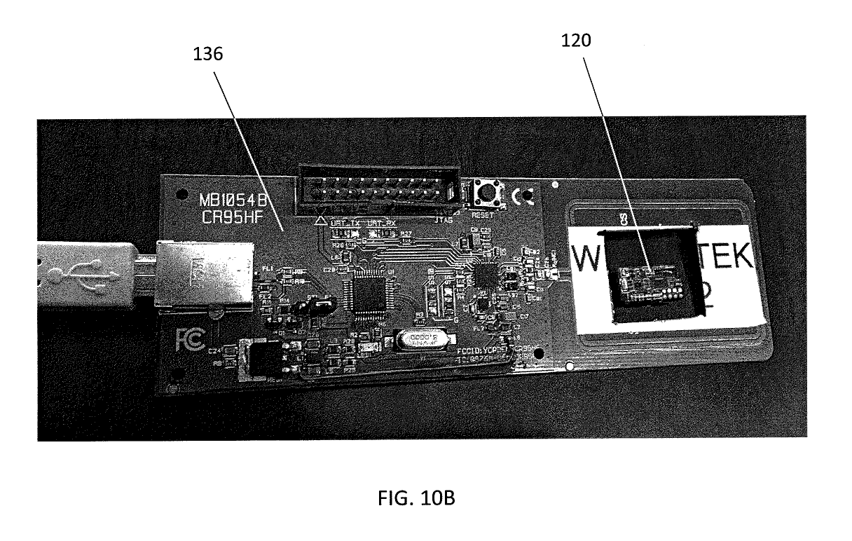

An exemplary schematic of a sensor unit is shown in FIGS. 10A and 10B. The printed circuit board (PCB) board layout shows circuitry for a simple processor that also measures temperature, the capacity for power, the charging circuitry for the capacitor, and the sensor unit pins for contact data/power source (where the base module plugs in). In some embodiments, the sensor unit can be smaller than shown in the figures.

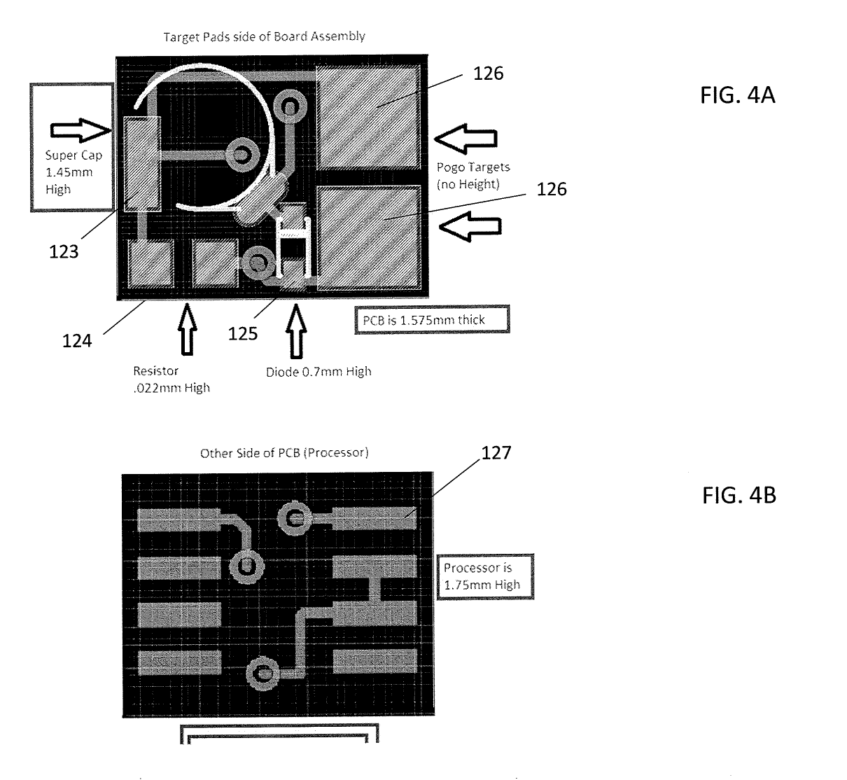

FIGS. 4A and 4B are depictions of the electronic components of a representative sensor unit 120. The sensor unit 120 can include a power source 123, such as a supercapacitor, a resistor 124, a diode 125, targets 126, and a microprocessor 127. In some embodiments, the power supply 123 works on low power, such as can run off of a current of less than 1 milliamp, such as less than 100 microamps, such as less than 10 microamps.

The sensor unit 120 can include one or more sensors 121 for measuring at least one parameter. Sensors 121 can be configured to detect or measure a parameter related to a characteristic of the environment it is in, record the measured parameter, and transmit the recorded parameter set to a receiving device. The sensors 121 may be, for example, motion, pressure, or positional sensors, and/or any other sense that is able to measure a biological value or a value associated with the oral environment. Motion or positional sensors may be used to monitor any changes to the position of the teeth during orthodontic adjustment. In those instances, the sensors 121 may be equipped with linear accelerometers that are coupled to orthodontic brackets and tracks movement over time. Other potential positional sensors 121 may include: capacitive transducers, capacitive displacement sensors, Eddy-current sensors, ultrasonic sensors, grating sensors, Hall effect sensors, inductive non-contact position sensors, laser Doppler vibrometer, linear variable differential transformer, multi-axis displacement transducer, photodiode arrays, piezo-electric transducter, potentiometers, proximity sensors, and rotary encoders. In the instance where positional sensors are employed, the sensors may be equipped with accelerometers, and/or original equipment manufacturer (OEM) based GPS modules. Pressure sensors detect pressure and may include, but not limited to absolute pressure sensors, gauge pressure sensors, vacuum pressure sensors, differential pressure sensors, and sealed pressure sensors. Some pressure sensors are force type sensors that collect a force value to measure strain when pressure is applied to the area and include piezo resistive strain gauge, capacitive, electromagnetic, piezoelectric, optical, and potentiometric.

Other parameters that may be detected by the sensors 121 include the physical condition of the environment, including temperature, pH, body position, teeth grinding (Bruxism), oxygen concentration/emission, carbon dioxide concentration/emission, bacterial count, and so forth. The sensors 121 described herein can be configured measure a biological parameter, such as glucose level, heart rate, arrhythmias, and so forth. In some instances where the sensors are used to measure a parameter from fluid (such as measuring glucose level or bacteria count in saliva), the corresponding sensors 121 are configured to allow fluid to channel into a compartment where an electrode can measure strength of an electrical signal calibrated for the particular parameter, be it glucose level or bacteria count. The compartment into which the fluid is diverted for measuring the parameter of interest should be small enough that it prevents tissue, particulates (such as from food), and the wearer's tongue from entering. In some instances, a single sensor can be configured to measure more than one environmental parameter.

In other instances where sensors 121 may be able to determine heart rate, the sensor may be equipped with electrodes that can detect electrocardiogram (EKG) readings or pulses from arteries or arterial located in the palate, and the signal detected may be transformed into a heart rate reading.

In yet another instances where airflow, oxygen concentration, or carbon dioxide emissions are measured, the sensors 121 may be both carbon dioxide and oxygen sensors. The sensor may also be equipped with to utilize readings from a pitot-tube designed into the sensor 121 to measure airflow. Such a sensor may be positioned at the wearer's palate.

In some examples, the sensor unit 120 may contain a single sensor 121 to measure multiple parameters, while in other cases, multiple sensors 121 may be needed to obtain a single parameter. For example, the same sensor may be used to measure pH and glucose level by measuring the voltages between two electrodes, and converting the detected value into a pH value. The same sensor unit may be applied to measuring bacterial count in the sample by utilizing electrodes to measure electrical signals or using spectroscopic measurements (such as those from mass spectrometry) as a tool in accessing likelihood of gum disease. In other instances, there may need for multiple sensors to obtain values on one parameter. For example, for the sensor to measure teeth grinding, the sensor may be equipped with a combination of force, pressure, and/or strain gauges. Forces and movement generated by teeth clenching can be detected using these sensors or gauges.

In some embodiments, the sensors 121 obtain voltage readings. The voltage readings can be stored within the memory 122 of the sensor unit 120. As described further below, the voltage readings can then be transmitted to the base module 130, which can determine temperature readings and calibrate the data.

The sensor unit 120 (which can be replaced by sensor unit 220 in any embodiment described herein) can be coupled to the dental appliance 110, as shown in FIGS. 6 and 7. The sensor unit 120 may be attached or mounted to dental appliance 110 through bonding materials such as glues, resins, epoxy, and so forth. In some embodiments, the sensor unit 120 is mounted within a pocket formed in the dental appliance 110. In other embodiments, the sensor unit 120 is attached to the dental appliance 110 through clips or other attachment mechanisms.

Because the sensor unit 120 is intended to be used within a wearer's mouth, it can be important that the sensor components remain isolated from the wearer's oral environment, especially if the configuration where the sensor unit 120 is bonded to the dental appliance 110 and largely exposed to oral environment. In the bonded configuration, the sensor unit 120 may be hermetically sealed. This is not only to protect the wearer from the potential harmful effects of leeching chemicals from the sensor components, but also to protect the sensor components from the warm, moist environment of the mouth. The sensor unit 120 may be sealed with any suitable material that is able to provide a sealed protective coating. Suitable coatings may be silicon-based compounds, ceramic-based compounds, plastic materials, resins, resinoids, polymers, cellulose derivatives, casein materials, protein-based coating, and/or composite materials. The proposed embodiments of such device can be encapsulated or potted with a material considered biocompatible in the event of ingestion. Additionally, the hardware used to create the sensor, such as the battery, can be configured to be suitable to enter the body in the event that the encapsulation is compromised. One exemplary potting material is EPO-TEK 301-2, or any equivalent epoxy or resin.

Sensor unit 120 may be placed in or on various regions of the dental appliance 110. In some embodiments, the sensor unit 120 may be embedded within the dental appliance 110. This embodiment offers an extra layer of protection of the sensor unit 120 from the moisture of the oral environment and protecting the wearer from potentially harmful chemicals contained within the electronic components because the sensor unit 120 is embedded within the dental appliance 110 (or 210). The placement of the sensor unit 120 may be in any location on the dental appliance 110 that is the least obtrusive to the wearer.

Two possible sensor positions are shown in FIGS. 6 and 7. FIG. 6 shows sensor unit 120 attached to dental appliance 110, while in FIG. 7, sensor unit 220 is embedded within dental appliance 210. In FIG. 6, the sensor unit 120 is attached on the outer face of the dental appliance 110 at a location corresponding to the rear molars. This position has the advantage that it corresponds to a void or dead zone within the oral cavity and it is also a location where it is difficult for the wearer's tongue to reach. The sensor unit 220 in the embodiment shown in FIG. 7 is embedded in the portion of the retainer that sit adjacent to the wearer's palate. The advantage of this embodiment is that the sensor unit 220 is essentially out of the way and there is less likelihood that the sensor unit 220 will become detached from the dental appliance 210 during use. Finally, while only two sensor positions are shown here, other viable sensor placement on the dental appliance has been contemplated, such as on an inner surface of the dental appliance or coupled to a wire portion of the orthodontic retainer.

Sensor unit 120, as a whole, can include one or more sensing elements, memory components for storing the parameter values detected, wireless transmitters, and a rechargeable battery. In some embodiments, the parameters detected are stored in volatile memory where once the information has been transferred to the base module 130, the information is deleted. In other embodiments, some of the information detected or registered by the sensor are maintained in non-volatile memory, such as calibrations for various parameter to be measured at a future date.

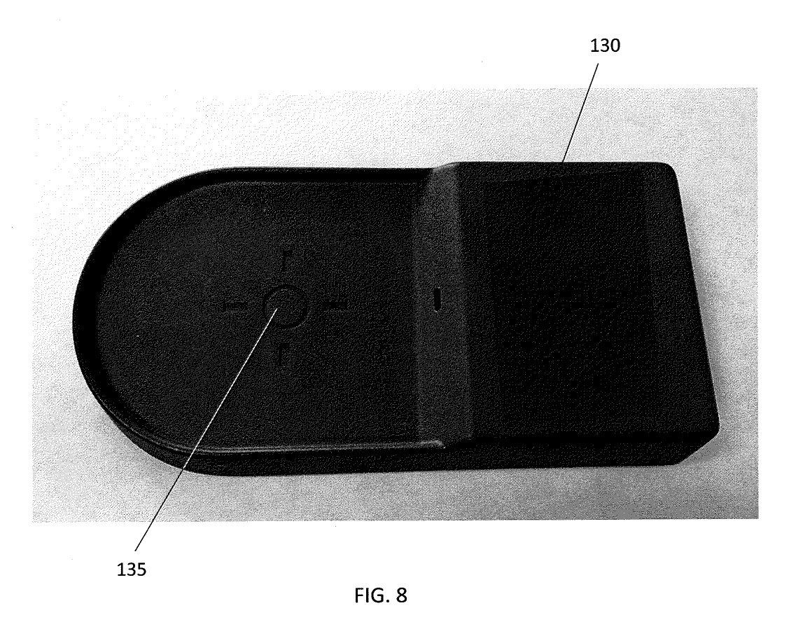

Sensor unit 120 can also include components for wireless transmitting parameter values measured to the base module 130. In order to accomplish this, sensor unit 120 can be placed on the flat, receiving surface of the base module 130. A sample embodiment of the base module 130 is shown in FIG. 8. Base module 130 includes electronic components such as processors 130, memory 132, sensors 133, and storage 134. Processor 130 is configured to manage the parameter data received from the sensor or sensors 120. Memory 132 is configured to store the data received from sensor unit 120 until it is transmitted to an analyzer 140, where analyzer 140 is contained within a telecommunication device such as a computer, tablet, or smart phone. Base module 130 may also contain sensor for detecting the presence of sensor unit 120 when sensor unit 120 is in close vicinity of the base module 130. Finally, base module 130 may also contain electronic components for volatile and non-volatile memory storage.

Even though it is possible for sensor unit 120 to be on continuously, it may be more efficient if sensor unit 120 only periodically turns on for detecting and recording the desired parameter or parameters. Having sensor unit 120 only turn on at pre-defined times will save on battery life of sensor unit 120. In some embodiments, sensor unit 120 may be “awakened” based on sensing a pre-defined magnitude of change in the parameter or a combination of parameters being measured. In other embodiments, sensor unit 120 may be configured to “awaken” once it is removed from base module 130. Once dental appliance is placed in the wearer's mouth, sensor unit 120 may be configured to taken readings periodically until the pre-defined magnitude of change in the parameter or parameters being measured is again detected or the sensor 110 is within a predetermined distance from the base module 130. Periodic readings may be every 5 minutes, every 10 minutes, every 15 minutes, every 20 minutes, and so forth. In some embodiments, the sensor unit 110 and the base module 130 can be configured to communicate information only when the sensor unit 120 (and thus the dental appliance) is within a set distance from the base module 130, e.g., is sitting on top of the base module. In some embodiments, the sensor unit 110 is prevented entirely from communicating with the base module when the dental appliance is in the mouth.

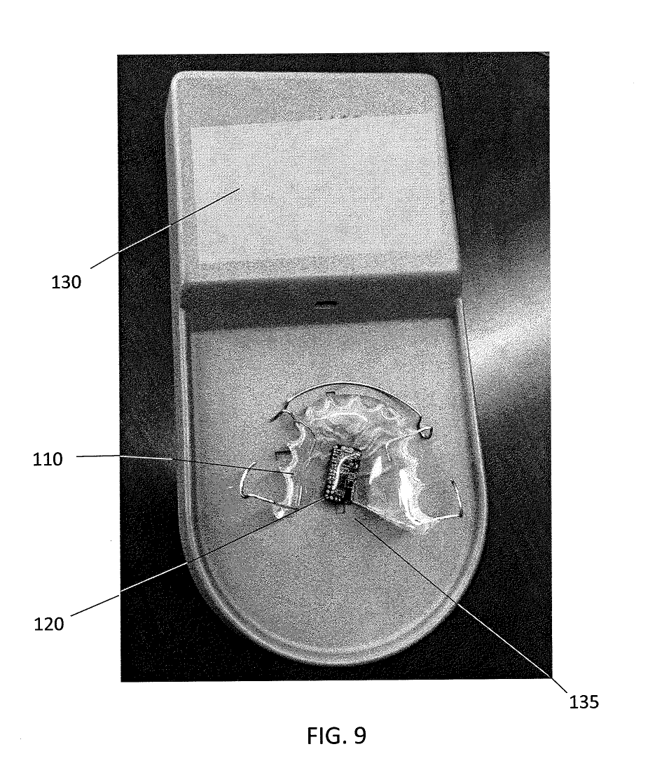

One embodiment of base module is shown in FIGS. 8-10B. The exterior of base module 130 is shown in FIG. 8. Base module 130 includes an optimal charging mark 135. By placing dental appliance 110 with coupled or embedded sensor unit 120 on charging mark 135, base module 135 will be able to charge sensor unit 120, and sensor unit 120 is able to transfer any recorded data to base module 135. An example of this is shown in FIG. 9 where base module 135 may recharge sensor unit 120 through inductive charging. In other examples, base module 130 may recharge sensor unit 120 using other wireless technology including, but not limited to RFID, Near Field Communication (NFC), proprietary RF protocols, and infrared communication. Base module 130 may also include a USB port, which can be an alternative method of transferring data to and from the analyzer 140. The base module 130 may also include a corded means for receiving power from a wall outlet or may be battery-powered. FIGS. 10A and 10B both show pictorials of internal electronic components 136 for driving the base module 130. The electronic components include an antennae and microcontroller that are able to interact with the sensor unit 120 when it is in the sweet spot 135 of the base module 130. In other examples, the base module 130 through its antennae is able to sense sensor unit 120 when the sensor unit 120 is below, above, or situated anywhere on the base module 130.

Referring back to FIGS. 1 and 2, the dental usage monitoring system 100 includes an analyzer 14, which can include programs and applications that are able to process and display the data detected, recorded, and transmitted from sensor unit 120 to base module 130 and finally to analyzer 140. Analyzer 140 can also include methods for accessing, processing, and transforming imprecise sensor data through discrete decisions. The analyzer 140 can process the stored and calibrated data to come to a discrete decision (such as the amount of time a retainer was worn or whether a user suffers from sleep apnea).

As shown in FIG. 2, the sensor unit 120 can include one or more sensors that can detect and measure environmental, network, or other metrics while storing the measured values in memory with a timestamp. In some embodiments, the sensor unit 120 can be small and battery operated. The sensor unit 120 can be designed to meet various criteria, including long battery life, ultra-small form factor, and/or safe human consumption that will impact the selection of sensor. Some sensors, such as temperature sensors, might have imprecise data when read due to fluctuations in supplied voltage when a battery or capacitor is nearly discharged. These issues lead to sensor data that may be imprecise when the value is read but given enough knowledge of how the design criteria will impact the imprecision, an intelligent system as described herein is able to correct, transform, and make decisions on these readings.

As shown in FIG. 2, the base module 130 can include a series of sensors 133 that can be used to measure the relative difference to the local and sensor readings, a microprocessor 130 to perform arithmetic and other complex operations on captured data, and a memory and storage to store and forward captured sensor data. The base module 130 can be designed to calibrate the system for the designed imprecise sensors. For example, the base module can measure ambient temperature when the sensor unit is plugged in, which can then serve as a baseline reading that indicates how inaccurate the sensor's reading is from actual. The base module 130 can be configured to calibrate based upon this initial data as well as data gathered throughout the use of the sensor. For example, if a temperature reading is lower than expected, but the voltage is also low (indicating that the temperature reading may be low as a result of low voltage), the base module can adjust the resulting temperature data to account for the error. The calibration performed by the base module will not involve use of a simple arithmetic difference, as each sensor will have a different behavior when issues, such as drop in voltage, occur. Rather, a complicated calibration algorithm can be implemented. The base module can store the calibration data with the time-series data into local memory for later transmission.

Referring still to FIG. 2, the analyzer 140 can include a microprocessor 141 to perform complex analysis on data forwarded from the base module 130, memory 142 to store the data from many base modules, a decision classifier 143 for performing machine assisted analysis on large amounts of sensor data, and a User Interface 144 to display captured data and offered an interactive interface to the decision classifier. The analyzer 140 can extract information from the base module 130 and perform several operations to transform the data set into a usable form to make decisions. This transformation involves shaping the incoming time series data with the calibration matrix, adding in known facts about the sensor (perhaps the sensor only measures temperature from 40-90° C. and thus drops several bits of data during transmission), running a transform algorithm (wavelet) on the data, and finally using a decision classifier to interpret the results. This gives the end user the ability to read raw sensor data that might not be useful outside of the time series context, and ask the system to make a decision on selected data. An example of this decision is if a temperature reading sensor is inside the human body. The sensor might not be reading 98.6 degrees F. all the time, but it can interpret the change from calibration sets into changes in the time series data to have a high certainty that the sensor is inside the human body or not. For example, in one embodiment, the sensor unit can be part of an orthodontic retainer, and the analyzer can be configured to consider calibrated temperatures of between 90 and 100 degrees F. as indicating that the sensor is within the mouth and calibrated temperatures of below that range as indicating that the sensor unit (and thus the dental appliance) is out of the mouth.

In some embodiments, the analyzer 140 can determine a total length of time and/or total periods of time of wearing the dental appliance based upon measurements taken by the sensor at discrete times. In one embodiment, the analyzer 140 can use a tree classifier to do so.

The analyzer 140 described herein may be a program that may be installed onto a user's computer or laptop. There may also be corresponding applications that can be installed into the user's tablet or smart phone.

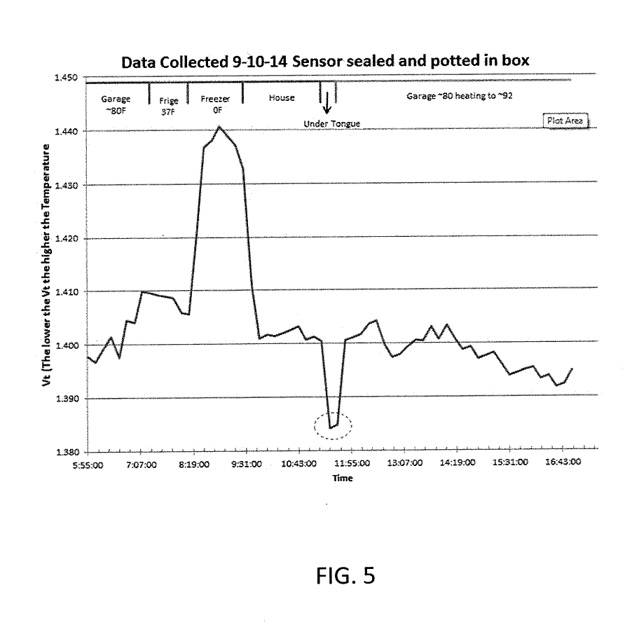

FIG. 5 is a graph showing voltage measurements taken by a sensor. As can be seen, the sensor 121 is able to detect changes in voltage where the extreme positive peaks and the negative peaks correspond to a drop in temperature (sensor placed in a freezer) and the sensor being placed under the tongue, respectively. In this embodiment, the sensor unit is a voltage sensor configured to sit within the mouth, such as between the teeth and the gums or on the roof of the mouth. For example, the sensor unit can be configured as part of an orthodontic retainer. The sensor data can be correlated to temperature readings by the base module, and that data can then be transmitted to the analyzer. The resulting voltage versus time graph of FIG. 5 shows someone placing the sensor unit in a warm environment, followed by a cooler environment, freezing environment, and then in the mouth. These valley circled “under tongue” shows a valley in the calibrated readings that shows it is inside the mouth. When it is removed and placed in a hot area, it is clearly visible. The analyzer picks up on this valley and measures the time series data to be “in the mouth” or not. In some embodiments, this type of temperature data can be gathered and used to determine whether the retainer or other orthodontic device was used as intended or prescribed by the orthodontist.

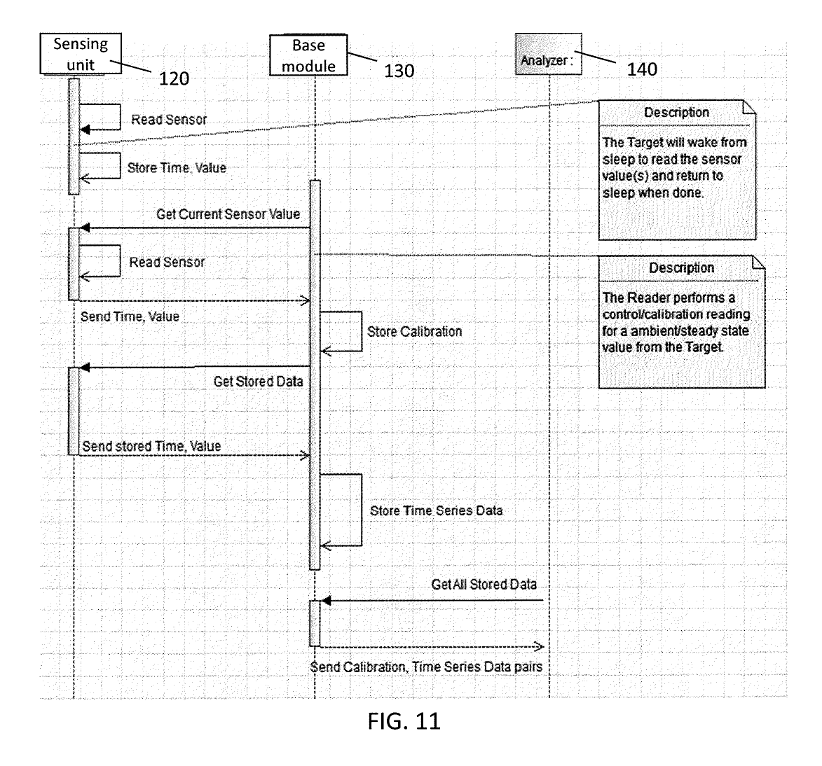

The relationship between the three components (the sensor unit, base module, and analyzer) of the sensor unit described herein are shown in FIG. 11. The sensor unit is shown, at a minimum, reading sensor values and storing the values with a timestamp. The sensor unit can perform this action periodically or based on some internal or external trigger. The base module, when connected to the sensor unit wirelessly or via a wire, requests a current sampling of the sensor data and compares the value(s) with the internal sensor readings. These values are compared and form the basis of an internal calibration to detect sensor reading drift, variance, or any other correctable anomaly. Once the calibration is determined, the base module will capture all stored data from the sensor unit's memory. The sensor unit's data is stored in the base module's long term storage with previous and future sampling of sensor unit data and may save multiple sensors worth of data. This stored data in the base module will then be sent to the analyzer wirelessly or via a wire and may or may not be purged from the base module's memory.

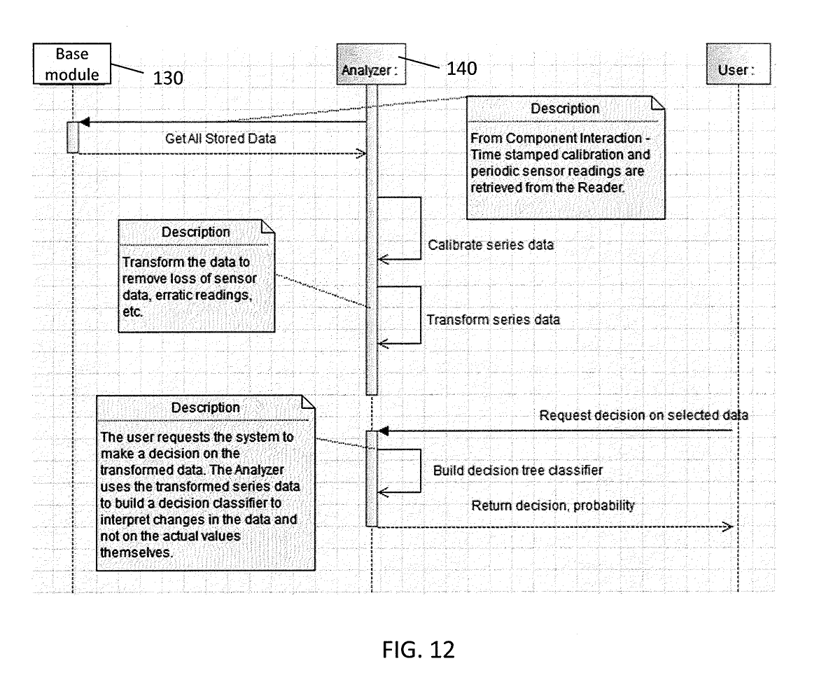

The relationship between the base module 130 and analyzer 140 is shown in FIG. 12. The base module transmits a signal to the analyzer 140 that data is ready for analysis and the analyzer 140 will request to get the stored sensor unit readings. Once the analyzer 140 has the stored data, it can calibrate the series data based on known calibration data points for the current data read from the base module or based on a priori calibration data and transformed to remove invalid sensor data as it pertains to the time series data. The user interacts with the analyzer 140 to request the system make a decision based on a portion or all of the transformed data. These decisions are based on an internal classifier built for the specific need such as determining if the sensor values indicate a specific trend or have a high probability of matching a specific decision.

The proposed device or other embodiments of such device can be configured to communicate wirelessly to mobile phones/mobile phone applications to convey treatment progress, location of misplaced retainers or aligners, or any sort of notification regarding the sensor unit. In the event that the retainer/aligner is separated from the user's phone, a text message or alert can be delivered to notify the user that they have misplaced or left behind their retainer/aligner. Additionally, statuses such as low battery, device fully charged, device in use, and device connected to base module should be available when accessing the mobile application.

The proposed device or other embodiments of such device can be configured to connect to a computer and through the base module and interact with the user using a graphical user interface (GUI). This GUI will allow the user and physician to view usage summaries and stored records, and for the physician only, allow assignment of the device to the patient.

In some embodiments, all three components (the sensor unit, base module, and analyzer) can be part of a single encapsulated device.

In some embodiments, the sensor can include a GPS sensor therein. The base module and/or analyzer can receive information from the GPS sensor to locate the sensor (and thus the dental appliance), such as if lost by the user.

In some embodiments, the sensor can be configured to activate or awaken to take measurements based upon readings from an accelerometer or other motion indicator.

Methods of using the dental usage monitoring system will now be described. Typically, a dental provider will provide the system to their patients for monitoring usage of the dental appliance. The patients will first fully charge the dental appliance having a sensor by placing the dental appliance on the base module. Once charged, the sensor will be able to detect a pre-set change in the parameter to be measured and when such a change is detected, the sensor will begin recording the parameter periodically until a second pre-set parameter value is detected or a pre-set time is met. The patient may then place the sensor coupled to the dental appliance on the base module to transmit raw sensor data to the base module, where the base module will store the raw data until it is able to be retrieved by the analyzer. The base module will also be able to recharge the sensor unit for subsequent use. The analyzer will then take the raw data and using appropriate algorithms, transform the raw, and potentially imprecise data (including missing or discrete datapoints) into data that can precisely determine the parameter detected, such as dental appliance usage.

### EXAMPLE 1

An exemplary algorithm for measuring temperature with a temperature sensor while accounting for data loss and voltage loss is shown below using HEX values:
The base module reader retrieves data as shown below and generates a graph. These values are eventually be fed into the decision classifier and transform for data interpretation. The communication between the analyzer and base module reader is through a USB to UART convertor in the reader right now but could also be WIFE Cellular, etc.
To determine the initial 2 MSBits, the analyzer sends a 3 to the base module reader, the base module reader returns the 10 bit readings for temperature and data. When the sensor data leaves the reader, this information allows determination of the 2 MSB its that is not stored.
Analyzer sends a request for target (sensor) status including temperature and voltage:
   Target returns—FE03023A0186FDC3
   FE is the start of header, 03 is the command it is responding to
   023A is Ntm
   0186 is Nsc
   FD is the end of header and C3 is the check character value (check character=N1+N2+N3+)
The base module reader sends a signal to erase old data
Target (sensor unit) is removed and starts collecting data
Target (sensor unit) module is put back in reader
Analyzer sends a request for the reader to acquire new sensor readings 1
Target (sensor unit) returns
   FE013D843B8638883689348A318C2F8D2D8F2B912A93289427962597239 9219B1F9C1E9E1D9F1BA119A318A516A615A8FFFFFFFFFFFFFFFFFFF FFFFFFFFFFFFFFFFFFFFFFFFFFFFFFFFFFFFFFFFFFFFFFFFFFFFFFFF FFFFFFFFFFFFFFFFFFFFFFFFFFFFFFFFFFFFFFFFFFFFFFFFFFFFFFFF FFFFFFFFFFFFFFFFFFFFFFFFFFFFFFFFFFFFFFFFFFFFFFFFFFFFFFFF FFFFFFFFFFFFFFFFFFFFFFFFFFFFFFFFFFFFFFFFFFFFFFFFFFFFFFFF FFFFFFFFFFFFFFFFFFFFFFFFFFFFFFFFFFFFFFFFFFFFFFFFFFFFFFFF FFFFFFFFFFFFFFFFFFFFFFFFFFFFFFFFFFFFFFFFFFFFFFFFFFFFFFFF FFFFFFFFFFFFFFFFFFFFFFFFFFFFFFFFFFFFFFFFFFFFFFFFFFFFFFFF FFFFFFFFFD29
   FE is the start of header, 01 is the command it is responding to
   3D is Ntm missing 2 MSBits, 84 is Nsc missing the 2 MSB its, . . . FF means no data collected
   FD is the end of header and 29 is the check character value
Formulas:
   Ntm=A/D result measuring temp module (10 bits)
   Nsc=A/D result measuring Supply voltage (supercap=10 bits)
   VFSR is the internal ref voltage to measure supply voltage=1.024
   Vtemp=(VFSR/Nsc)*(1024−Ntm)=(1.024/Nsc)*(1024−Ntm)

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A dental usage monitoring system, comprising:
a dental appliance;
at least one sensor attached to the dental appliance and configured to collect data related to usage of the dental appliance; and
a power supply attached to the dental appliance and configured to operate the sensor using a current of less than 1 milliamp; and
an analyzer in communication with the sensor and configured to determine usage of the dental appliance based upon the collected data.

2. The dental usage monitoring system of claim 1, wherein the data is voltage data.

3. The dental usage monitoring system of claim 2, further comprising a base module configured to receive the collected data, determine temperature readings from the voltage data, and transmit the temperature readings to the analyzer.

4. The dental usage monitoring system of claim 3, wherein the base module is further configured to calibrate the collected data.

5. (canceled)

6. The dental usage monitoring system of claim 1, wherein the power supply is a supercapacitor.

7. The dental usage monitoring system of claim 1, wherein the sensor is embedded in the dental appliance.

8. The dental usage monitoring system of claim 1, wherein the sensor is attached to the dental appliance in a region that corresponds to an open or empty pocket between a user's teeth and a buccal region of the user's mouth when in use.

9. The dental usage monitoring system of claim 1, wherein the sensor further comprises a protective coating therearound.

10. (canceled)

11. The dental system of claim 1, wherein the data is temperature, motion, position, force, pressure, pH, oxygen concentration, carbon dioxide concentration, bacteria count, heartbeat, or presence of arrhythmias.

12. The dental system of claim 1, wherein the dental appliance is a retainer.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A dental usage monitoring system, comprising:
a dental appliance;
at least one sensor attached to the dental appliance, the sensor configured to collect data related to usage of the dental appliance at discrete timepoints; and
an analyzer in communication with the sensor and configured to determine total usage of the dental appliance based upon the data collected at discrete timepoints.

29. The dental usage monitoring system of claim 28, wherein the sensor is configured to take measurements only when activated.

30. The dental usage monitoring system of claim 29, wherein the sensor is configured to be activated based upon removal from a base module.

31. The dental usage monitoring system of claim 28, wherein the timepoints are at least 5 minutes apart.

32. The dental usage monitoring system of claim 28, wherein the analyzer is configured to use a decision tree classifier to process variability in the data received and to determine the total usage.

33. The dental usage monitoring system of claim 28, wherein the sensor is configured to transmit data to the base module only when the sensor is a set distance from the base module.

34. (canceled)

35. The dental usage monitoring system of claim 28, wherein the sensor is embedded in the dental appliance.

36. The dental usage monitoring system of claim 28, wherein the sensor is attached to the dental appliance in a region that corresponds to an open or empty pocket between a user's teeth and a buccal region of the user's mouth when in use.

37. The dental usage monitoring system of claim 28, wherein the sensor further comprises a protective coating therearound.

38. (canceled)

39. The dental system of claim 28, wherein the data is temperature, motion, position, force, pressure, pH, oxygen concentration, carbon dioxide concentration, bacteria count, heartbeat, or presence of arrhythmias.

40. The dental system of claim 28, wherein the dental appliance is a retainer.

41. (canceled)

42. (canceled)

43. (canceled)

44. (canceled)
